(19) United States
(12) Patent Application Publication
(10) Pub. No.: US 2025/0152765 A1
(43) Pub. Date: May 15, 2025

(54) DEVICE AND METHOD FOR THE GENERATION, CONTROL, AND APPLICATION OF HYDROGEN PEROXIDE VAPOR DOSE

(71) Applicant: Ján SOFRANKO, Siroke (SK)

(72) Inventor: Ján SOFRANKO, Siroke (SK)

(21) Appl. No.: 18/834,972

(22) PCT Filed: Jun. 16, 2022
(86) PCT No.: PCT/IB2022/055581

§ 371 Date: Jul. 31, 2024

(30) Foreign Application Priority Data
Jun. 6, 2022 (SK) PP-59-2022

(51) Int. Cl. A61L 2/20; C01B 15/01

(52) U.S. Cl. CPC A61L 2/208; C01B 15/01; A61L 2202/11

(57) ABSTRACT

A device for the generation, control, and application of a hydrogen peroxide vapor dose includes an injection chamber (1), an inlet valve (10), an outlet valve (11), a chamber depressurization valve (9), an injection chamber pressure sensor (3), an injection valve (2), a hydrogen peroxide supply line (16), a carrier medium supply line (13), and an application line (15). A required amount of liquid hydrogen peroxide (8) is injected onto the hot injection surface of the chamber (12) of the closed, heated, and depressurized injection chamber (1) via the injection valve (2), thereby changing the state of hydrogen peroxide from liquid to gas. The correctness of the injected amount of liquid hydrogen peroxide (8) is evaluated by measuring the pressure rise in the injection chamber (1), and a required batch of hydrogen peroxide vapor is generated at a desired concentration.

### FIELD OF TECHNOLOGY

The invention relates to a method for the generation, control, and application of small and precise doses of hydrogen peroxide vapor intended for sterilization of objects, equipment, and premises.

### PRIOR ART

At present, the objects and premises are sterilized by the continuous hydrogen peroxide vapor generators, which allow for the continuous vapor generation, without recording the generated amount of vapor.

### ESSENCE OF THE INVENTION

The device and method for the generation, control, and application of hydrogen peroxide vapor dose according to the present invention addresses the generation, control, and application of hydrogen peroxide vapor for the sterilization purposes, where small and precise doses of hydrogen peroxide vapor are required. The device produces a vapor dose by injecting the exact amount of liquid hydrogen peroxide onto the hot surface of the injection chamber, with various parameters per each dose of vapor. After the liquid hydrogen peroxide is injected, the pressure in the injection chamber increases, the value of which indicates the content of the required amount of hydrogen peroxide in the generated hydrogen peroxide vapor dose. The device allows producing individual doses of vapor in different concentrations of hydrogen peroxide.

### OVERVIEW OF FIGURES IN THE DRAWINGS

FIG. 1 shows the device for generating, controlling, and applying a hydrogen peroxide vapor dose. It is also a picture for annotation.

FIG. 2 shows an injection valve.

### EXAMPLES OF EMBODIMENTS OF THE INVENTION

It is to be understood that the device construction according to the invention is presented by way of illustration and should not be construed as limiting its technical designs. Those skilled in the art will be able to ascertain many equivalents to the specific embodiments of the invention by applying no more than routine experimentation. Such equivalents of the constructed device and its parts will also fall within the scope of the patent claims. It cannot be problematic for those skilled in the art to optimally design the structure and select its elements; therefore, these features have not been addressed in this description in detail.

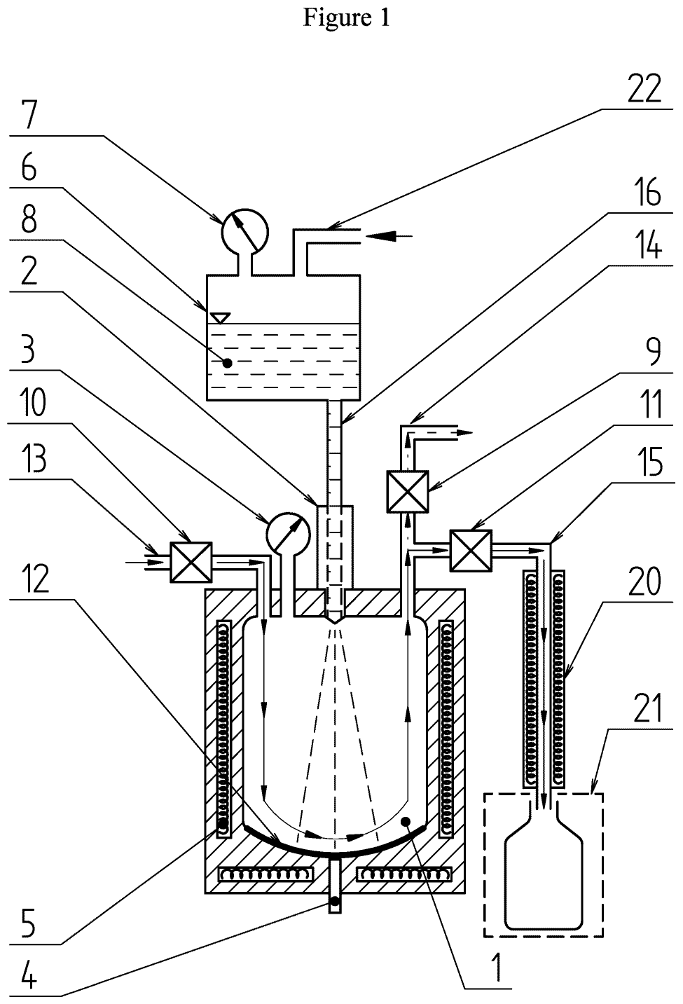

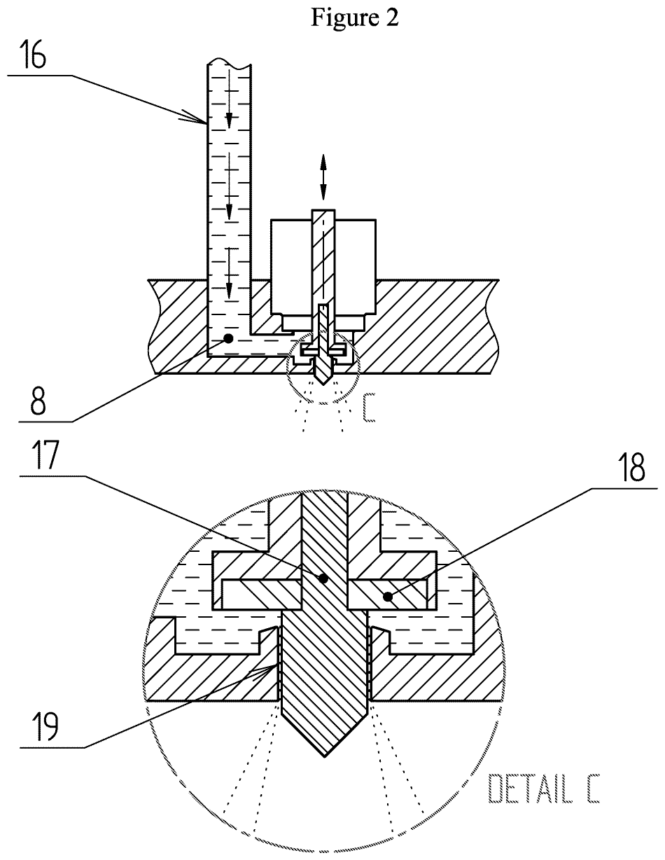

The device and method for generating, controlling, and applying a peroxide vapor dose shown in FIGS. 1 to 2 are formed by an injection chamber 1, an inlet valve 10, an outlet valve 11, a chamber depressurization valve 9, an injection chamber pressure sensor 3, an injection valve 2, a hydrogen peroxide supply line 16, a carrier medium supply line 13, and an application line 15. The injection chamber 1 is designed as a pressure-sealed vessel with heating of the injection chamber 5, and the injection chamber temperature sensor 4. The injection valve 2 is arranged in the injection chamber 1, opposite to which the chamber injection surface 12 is formed. The injection valve 2 is terminated by an injection valve nozzle 19 with an injection valve needle 17 and an injection valve seat 18. The device also includes a liquid hydrogen peroxide pressure tank 6 with a liquid hydrogen peroxide pressure sensor 7, and an application line 15.

The operating cycle of the device according to the invention begins with the opening of the chamber depressurization valve 9, whereby the pressure of the heated injection chamber 1 equalizes with the ambient pressure through the depressurization line 14. After the pressure set point has been recorded by the injection chamber pressure sensor 3, the chamber depressurization valve 9 closes, sealing the injection chamber 1 in pressure.

In the liquid hydrogen peroxide pressure tank 6, a pressure higher than that in the injection chamber 1 is being maintained above the liquid hydrogen peroxide level 8 by the compressed air supply line for the peroxide tank 22. By opening the injection valve 2, the required amount of liquid hydrogen peroxide 8 is injected into the injection chamber 1. The injection of the liquid hydrogen peroxide 8 on the hot injection surface of the chamber 12 changes the hydrogen peroxide state from liquid to gaseous, and the desired dose of hydrogen peroxide vapor is formed at the desired concentration. After the liquid hydrogen peroxide 8 is injected onto the hot injection surface of the chamber 12, the pressure in the injection chamber 1 rises. By measuring this pressure increase by the pressure sensor of the injection chamber 3, the device according to the invention evaluates whether the required amount of liquid hydrogen peroxide 8 has been injected.

If the device according to the invention evaluated that the correct amount of liquid hydrogen peroxide 8 has been injected, the device continues in the prescribed operating cycle. By opening the inlet valve 10 and the outlet valve 11, the carrier medium begins to flow into the injection chamber 1 through the carrier medium supply line 1, and fills the injection chamber 1 with the new carrier medium; this medium pushes the produced dose of hydrogen peroxide vapor through the outlet valve 11 and the application line 15 to the desired site of application of the peroxide vapor 21.

If another dose of peroxide vapor is to be produced, the duty cycle is repeated.

If the device according to the invention evaluated that the wrong amount of liquid hydrogen peroxide 8 has been injected into the injection chamber 1, the operating cycle starts from the beginning.

The change in the dose density of the peroxide vapor produced by the device according to the invention can be achieved by changing the individual parameters for the liquid hydrogen peroxide 8 injection into the injection chamber 1, namely the injection pressure of liquid hydrogen peroxide 8, the length of injection valve 2 opening, the temperature of the injection chamber 1, and the temperature of the chamber injection surface 12, the pressure of the carrier medium, with which the injection chamber 1 is flushed, and the length of flushing of the injection chamber 1.

It is particularly important to maintain a correct and constant temperature of the chamber injection surface 12. Since liquid hydrogen peroxide 8 is injected onto this chamber injection surface 12, the chamber injection surface 12 cools rapidly and, therefore, requires special access to heating.

For the proper application of hydrogen peroxide vapor to the desired peroxide vapor site of application, it is necessary to maintain the correct temperature of the application line 15 and of all surfaces of the piping and valves that come in contact with the hydrogen peroxide vapor.

Due to its construction and the method of generation, control, and application of the vapor dose, the device is also suitable for the generation of vapors from other liquid substances and compounds than hydrogen peroxide alone.

### DESCRIPTION OF TERMS

Carrier medium—Compressed gas (e.g. air, nitrogen)
Inlet valve—Closes the supply line of the carrier medium
Outlet valve—Separates the injection chamber from the application line
Chamber depressurization valve—Separates the injection chamber from the outside environment
Sterilization—A process, by which all microorganisms in a given environment, object or equipment are destroyed—
Condensation—A physical process of converting a gaseous into liquid state

### Legend to the Attached Drawings—FIGS. 1 to 2

1) Injection chamber
2) Injection valve
3) Injection chamber pressure sensor
4) Injection chamber temperature sensor
5) Heating of the injection chamber
6) Liquid hydrogen peroxide pressure tank
7) Liquid hydrogen peroxide pressure sensor
8) Liquid hydrogen peroxide
9) Chamber depressurization valve
10) Inlet valve
11) Outlet valve
12) Injection surface of the chamber
13) Carrier medium supply line
14) Depressurization line
15) Application line
16) Hydrogen peroxide supply line
17) Injection valve needle
18) Injection valve seat
19) Injection valve nozzle
20) Application line heating
21) Site of peroxide vapor application
22) Compressed air supply line for peroxide tank

1. The device for the generation, control, and application of a hydrogen peroxide vapor dose characterized in that it consists of an injection chamber (1), an inlet valve (10), an outlet valve (11), a depressurization valve (9), an injection chamber pressure sensor (3), an injection valve (2), a hydrogen peroxide supply line (16), a carrier medium supply line (13), and an application line (15).

2. The device for the generation, control, and application of a hydrogen peroxide vapor dose characterized in that the injection valve needle (17) moves within the injection valve nozzle (19), when the injection valve (2) opens and closes, thereby mechanically cleaning the walls of the injection valve nozzle (19).

3. A method for the generation, control, and application of a hydrogen peroxide vapor dose by the device according to the invention characterized in that the injection valve (2) injects the required amount of liquid hydrogen peroxide (8) onto the hot injection surface of the chamber (12) of the closed, heated and depressurized injection chamber (1), thereby changing the state of hydrogen peroxide from liquid to gaseous; then, the correctness of the injected amount of liquid hydrogen peroxide (8) is evaluated by measuring the pressure rise in the injection chamber (1), and the desired dose of hydrogen peroxide vapor is generated at a desired concentration.
